SEQUENCE LISTING (14 sequences)

SEQ ID NO: 1 - protein, 15 aa, Homo Sapiens
EPKSCDKTHTCPPCP

SEQ ID NO: 2 - protein, 12 aa, Homo Sapiens
ERKCCVECPPCP

SEQ ID NO: 3 - protein, 61 aa, Homo Sapiens
ELKTPLGDTTHTCPRCPEKSCDTPPPCPRCPEPKSCDTPPPCPRCPCPKSCDTPPPCPRCP

SEQ ID NO: 4 - protein, 12 aa, Homo Sapiens
ESKYGPPCPSCP

SEQ ID NO: 5 - protein, 110 aa, Homo Sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 6 - protein, 110 aa, Homo Sapiens; identical to SEQ ID NO: 5

SEQ ID NO: 7 - protein, 110 aa, Homo Sapiens
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTK

SEQ ID NO: 8 - protein, 110 aa, Homo Sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 9 - protein, 107 aa, Homo Sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 107 aa, Homo Sapiens
GQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 11 - protein, 107 aa, Homo Sapiens
GQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 12 - protein, 107 aa, Homo Sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 7 aa, Homo Sapiens
TLVTVSS

SEQ ID NO: 14 - protein, 269 aa, Artificial Sequence [sequence omitted]

(19) United States
(12) Patent Application Publication
(10) Pub. No.: US 2009/0306349 A1
(43) Pub. Date: Dec. 10, 2009

(54) BINDING PARTNERS WITH IMMUNOGLOBULIN DOMAINS MODIFIED TO HAVE EXTENDED HALF-LIFE

(75) Inventors: Maria U. Hutchins, Mountain View, CA (US); Samuel Zalipsky, Redwood City, CA (US); Bernard J. Scallon, Wayne, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: Rampak Corp

(21) Appl. No.: 12/295,334

(22) PCT Filed: Mar. 30, 2007
(86) PCT No.: PCT/US07/65610

§ 371 Date: Jul. 6, 2009

Related U.S. Application Data
(60) Provisional application No. 60/788,182, filed on Mar. 31, 2006.

(51) Int. Cl. C07K 16/00; C07K 1/107

(52) U.S. Cl. 530/391.1; 530/410

(57) ABSTRACT

A method for conjugation of glycosylated Fc-containing proteins is described. The method comprises carbamate chemistry performed and neutral pH or below and the resulting Fc-containing protein are expected to retain tertiary structure and therefore Fc-related bioactivity such as FcR binding, the ability to bind Clq, in addition to retaining ligand binding capabilities related to the incorporation of a ligand binding peptide or other polypeptide which has binding specificity. The method is exemplified using an erythropoietin-mimetic peptide fused to a human IgG1 antibody constant domain comprising a hinge, CH2 and CH3.

### PRIOR APPLICATION

This application claims priority to U.S. application No. 60/788,182, filed Mar. 31, 2006 and PCT/U.S. 07/65610, filed Mar. 30, 2007, both of which are entirely incorporated herein by reference.

### FIELD OF THE INVENTION

The invention relates to methods of chemical modification of fusion proteins, which are characterized as having a recognition domain for a specific binding partner and immunoglobulin constant domains. In a preferred embodiment, the modification is an attached hydrophilic polymer, which confers extended serum half-life as compared to the parent fusion protein.

### BACKGROUND

Polyethylene glycol (PEG) is available in a range of molecular weights with several to thousands of ethylene glycol units. The polymer is also available with a variety of end-modifications for reaction with various biomolecules, for example, drugs and proteins. Indeed, reaction of biomolecules with PEG, often referred to as “PEGylation”, has become an accepted method to improve properties of biomolecules. Several PEGylated proteins are currently on the market (ADAGEN®, ONCOSPAR®, PEG-INTRON®, PEGASYS®, NEULASTA™, and SOMAVERT®) and additional PEGylated molecules are in clinical trials. PEGylation has generated much interest in the biopharma industry as a convenient means to prolong the blood circulation lifetime of protein products. Potential advantages of PEGylation include a resistance to proteolysis, improved solubility, and reduced aggregation. In addition, PEG conjugation of proteins often decreases the protein's immunogenicity and can improve tolerogenicity. PEGylation of proteins has also been used to enhance pharmacokinetic half-life (PK) of the protein.

Biologic molecules generally have a serum residence time that is a function of their molecular weight, with peptides having a half-life (t½) of minutes, while globular molecules may have a t½ of days or weeks. Antibodies, which are complex heterodimeric structures of about 150 kDa, contain unique highly conserved domains known as constant domains which impart non-antigen binding functions to the molecules and which play a role in increasing circulation t½ which are distinct from size alone. Due to their conserved nature and long-circulation time, the addition of these domains to other proteins has proved to be another strategy for increasing biological therapeutic t½ while providing a non-immunogenic molecule (U.S. Pat. Nos. 5,116,964 and 5,428,130; WO00/024782). Specialized fusion proteins of this type have also been successfully developed as therapeutics, e.g. Enteracept®, and other like molecules have been produced. A PEGylated chimeric glycoprotein molecule comprised of gp120-binding portion of human CD4 fused to the hinge of an Fc portion of human IgG has also been reported (Chamow et al., 1994)

The combined strategies of creating antibody-mimicking fusion proteins and modifying such proteins with PEG or another hydrophilic polymer is desirable. However, there remains a need for a process to produce polymer conjugated antibody-mimicking fusion polypeptides without significantly reducing the activity or safety of the parent molecule.

### SUMMARY OF THE INVENTION

The present invention comprises a method for increasing the circulating half-life of an Fc-containing protein by attaching a hydrophilic polymer, which method comprises: contacting the Fc-containing protein with an activated carboxylic acid of a hydrophilic polymer in the presence of an activating agent at pH of less than about 7.0 or neutral pH, and purifying said conjugated Fc-containing protein. The method provides for the enhancement of circulating half-life of Fc-containing proteins such as but not limited to antibodies, anti-target Ig proteins, and MIMETIBODIES while retaining Fc-domain associated biological functions including Fc-receptor binding, Protein A binding, Protein G binding, Clq binding, ADCC, and CDC. The activating agent is preferably a water-soluble, non-carboxylic, Bronsted acid of moderate acidity having the propensity to donate N- or O-linked proteins to the activated carboxylic acid polymer reagent.

The conjugated Fc-containing proteins produced by the method of the invention are also provided. In particular, an Fc-containing fusion protein comprising an erythropoietin mimic peptide (EMP-1) conjugated by the method of the invention is provided.

### BRIEF DESCRIPTION OF THE FIGURES

FIG. 1 depicts antibody IgG domains showing the relationship between the domains and the major designated cleavage fragments.

FIG. 2 depicts a MIMETIBODY™ showing the relationship of engineered polypeptide ligands and the basic IgG constant domains comprising the fusion protein.

FIG. 3 is the amino acid sequence of a complete human IgG1 heavy chain constant region including CH1, hinge, CH2 and CH3. The 28 lysine residues (Ks) are shown in bold.

FIG. 4 is the amino acid sequence of CNTO528 annotated to show the residues belonging to each of the components such as V1, peptide, linker, V2/J, hinge, CH2, and CH3.

FIG. 5 shows a basic reaction scheme for addition of PEG to a Mimetibody™ (MMB) using methoxy-polyethyleneglycol-nitrophenyl carbonate (mPEG-NPC) reagent.

FIGS. 6A and 6B show tracings from HPLC-SEC analysis of PEGylated mimetibodies, PEG_{30K}-CNTO0528, prepared according to the reaction scheme of FIG. 5, the SEC analysis done on the crude reaction material (FIG. 6A) and after purification (FIG. 6B).

FIGS. 7A and 7B show tracings from HPLC-SEC analysis of PEGylated mimetibodies, PEG_{30K}-CNTO528, prepared according to the reaction scheme of FIG. 5 but with different initial reactant amounts from the material of FIGS. 6A-6B, the SEC analysis done on the crude reaction material (FIG. 7A) and after purification (FIG. 7B).

FIG. 7C is an electrophoresis gel for the purified PEG₃₀ₖ-CNTO528 conjugate of FIG. 7B, stained with Coomassie blue stain for protein detection (left lane) and with iodine stain for PEG detection (right lane).

FIGS. 8A and 8B are chromatograms of PEG_{30K}-CNTO528 (FIG. 8A) and of CNTO528 (FIG. 8B) binding to a Protein A column.

FIG. 9 shows an analysis of a PEG_{30K}-CNTO528 conjugate by MALDI-TOF MS.

FIG. 10 is an SDS-PAGE gel stained with Coomassie blue stain for protein detection of CNTO528 in conjugates of CNTO528 and PEG, the left lane corresponding to a PEG₃₀ₖ-CNTO528 with a relatively low level of PEGylation, the center lane conjugate corresponding to a PEG_{20K}-CNTO528 conjugate with a relatively low level of PEGylation, and the right lane corresponding to a PEG₃₀ₖ-CNTO528 conjugate with a relatively high level of PEGylation.

FIG. 11A is a graph showing the proliferation of an erythropoietin-dependent cell line, UT-7, incubated in the presence of CNTO528 (open squares) or in the presence of the conjugates of FIG. 10, specifically with PEG_{30K}-CNTO528 conjugate with a low level of PEGylation (open triangles) or with a higher level of PEGylation (x symbols), and with a PEG_{20K}-CNTO528 conjugate with a low level of PEGylation (+symbols).

FIG. 11B is a graph showing the relative extent of proliferation of an erythropoietin-dependent cell line, UT-7 after 24 hours, incubated in the presence of various concentrations of CNTO528 (open circles) or the PEG_{30K}-CNTO528 conjugate of Example 1 (* symbols) or the PEG₃₀ₖ-CNTO528 conjugate of Example 2 (open triangles) derived from FIG. 11A.

FIG. 12 is a graph of the serum concentration, in ng/mL, of Fc-containing protein over time, in days post injection, for rats injected once with either PEG_{30K}-CNTO528 with an average of 2 PEG per molecule or unconjugated CNTO528.

FIG. 13A is a plot of the average reticulocytes, in units of 10⁹ cells/L, as a function of time, in days, in the blood of rats dosed with saline (vertical line symbols) or with a single injection of increasing levels of either PEG_{30K}-CNTO528 with an average of 2 PEG per molecule or unconjugated CNTO528, where the rats treated with unmodified CNTO528 are represented by the open symbols (square, 25 mg/kg; triangle, 0.5 mg/kg; circle 1 mg/kg; X symbols, 2 mg/kg) and rats treated with the PEG-modified CNTO528 by the closed symbols (square, 0.5 mg/kg; triangle, 0.5 mg/kg; circle 1 mg/kg). Sham treated rats (injected with PBS only) are represented by a thin line and vertical tick marks.

FIG. 13B is a plot of the hemaglobin (Hb) values, in g/dL, as a function of time, in days, in the blood of rats dosed with saline or with a single injection of increasing levels of either PEG_{30K}-CNTO528 with an average of 2 PEG per molecule or unconjugated CNTO528, with the symbols and doses as set forth in FIG. 13A.

FIG. 13C is a plot of the hematocrit (Hct) values, in percent, as a function of time, in days, in the blood of rats dosed with a single injection of saline or of increasing levels of either PEG_{30K}-CNTO528 with an average of 2 PEG per molecule or unconjugated CNTO528, with the symbols and doses as set forth in FIG. 13A.

FIG. 13D is a plot of the average red blood cells, in units of 10⁶ cells/L, as a function of time, in days, in the blood of rats dosed with a single injection of saline or of increasing levels of either PEG_{30K}-CNTO528 with an average of 2 PEG per molecule or unconjugated CNTO528, with the symbols and doses as set forth in FIG. 13A.

### DESCRIPTION OF THE INVENTION

### Abbreviations

IgG, immunoglobulin G; PEG, polyethylene glycol; PK, pharmacokinetics; MMB, Mimetibody™; CNTO528, EPO, erythropoietin; NPC, nitrophenyl carbonate; HOSu, hydroxysuccinimate; Fc, crystallizable fragment; kDa, kilodalton; MMB, Mimetibody™; PK, pharmacokinetic; PD, pharmacodynamic; IEX, ion-exchange chromatography; Hb, hemoglobin; IV, intravenous; ELISA, enzyme-linked immunosorbant assay; PAGE, polyacrylamide gel electrophoresis; RP-HPLC, reversed phase high-performance liquid chromatography; SEC, size exclusion chromatography; MALDI-TOF-MS, matrix-assisted laser/desorption ionization time-of-flight mass spectrometry; ADCC, antibody-dependent cellular cytotoxicity; ATCC, American Type Culture Collection; BSA, bovine serum albumin; FBS, fetal bovine serum; PBS, phosphate-buffered saline; RT, room temperature; SDS, sodium dodecyl sulfate; PAGE, polyacrylimide gel electrophoresis.

### DEFINITIONS

The term “antibody” is intended to encompass antibodies, digestion fragments, specified portions and variants thereof, including, without limitation, antibody mimetics or comprising portions of antibodies that mimic the structure and/or function of an antibody or specified fragment or portion thereof, including, without limitation, single chain antibodies, single domain antibodies, minibodies, and fragments thereof. Functional fragments include antigen-binding fragments that bind to the target antigen of interest. For example, antibody fragments capable of binding to a target antigen or portions thereof, including, but not limited to, Fab (e.g., by papain digestion), Fab′ (e.g., by pepsin digestion and partial reduction) and F(ab′)₂ (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments, are encompassed by the term antibody (see, e.g., Colligan, et al., eds., Current Protocols in Immunology, John Wiley & Sons, Inc., NY (1994-2006). Thus, the term antibody, as used herein also includes antibody fragments either produced by the modification of whole antibodies or synthesized de novo using recombinant DNA methodologies

The terms “Fc,” “Fc-containing protein” or “Fc-containing molecule” as used herein refer to a monomeric, dimeric or heterodimeric protein having at least an immunoglobulin CH2 and CH3 domain. The CH2 and CH3 domains can form at least a part of the dimeric region of the protein molecule (e.g., antibody) when functionally linked to a dimerizing or multimerizing domain such as the antibody hinge domain. The Fc portion of the antibody molecule (fragment crystallizable, or fragment complement binding) denotes one of the well characterized fragments produced by digestion of an antibody with various peptidases, in this case pepsin. While various antibody fragments are defined in terms of the digestion of an intact antibody, one of skill will appreciate that such Fc fragments may be synthesized de novo either chemically or by utilizing recombinant DNA methodology, peptide display, or the like.

The term “monoclonal antibody” as used herein is a specific form of Fc-containing fusion protein comprising at least one ligand binding domain which retains substantial homology to at least one of a heavy or light chain antibody variable domain of at least one species of animal antibody and which binding domains have a specific and defined affinity for a ligand.

### Antibodies, Anti-Target Ig, and Other Antibody Mimics (Mimetibody™)

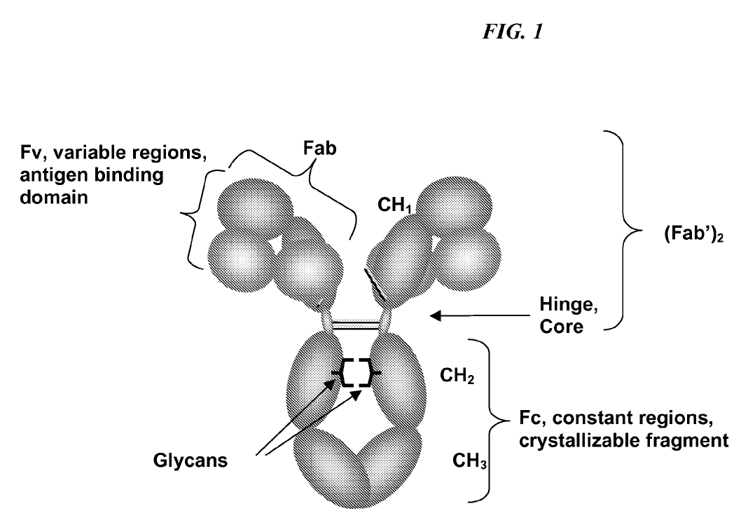

Among antibody classes (also known as isotypes) are IgE, IgD, IgA, IgM, and IgG. Antibodies are specialized immunoglobulin molecules which comprise a basic heterotetrameric glycoprotein of about 150,000 daltons, composed of two identical light (L) chains and two identical heavy (H) chains. Referring to FIG. 1, an antibody of the IgG class comprises two heavy chains and two light chains. Each chain has a constant region (C) and a variable region (V). Each chain is organised into a series of domains. The light chains have two domains, corresponding to the CL region and to the VL region. The heavy chains have four domains, one corresponding to the VH region and three domains (C1, C2 and C3) in the C region. Each heavy and light chain also has regularly spaced intrachain disulfide bridges. In human IgG antibodies, The constant domain of the light chain is aligned with the first constant domain of the heavy chain, and the light chain variable domain is aligned with the variable domain of the heavy chain. Particular amino acid residues are believed to form a noncovalent interface between the light and heavy chain variable domains (Clothia et al., J. Mol. Biol. 186, 651-66, 1985); Novotny and Haber, Proc. Natl. Acad. Sci. USA 82 4592-4596 (1985). The two heavy (H) chains are inter-connected by disulphide bonds at an interdomain region in the heavy chains known as the hinge.

IgGs are the most abundant with the IgG1 subclasses exhibiting the most significant degree and array of effector functions. IgG1-type antibodies are the most commonly used antibodies in cancer immunotherapy where ADCC and CDC activity are often deemed important. Structurally, the IgG hinge region and CH2 domains play a major role in the antibody effector functions. The N-linked oligosaccharides present in the Fc region (formed by the dimerization of the hinge, CH2 and CH3 domains) affect the effector functions. The covalently bound oligosaccharides are complex biantennary type structures, highly heterogeneous, and are covalently attached to the heavy chain at a conserved N-linked glycosylation site (NST) which lies within each CH2 domain. In the mature antibody, the two oligosaccharides are buried between the CH2 domains, forming extensive contacts with the polypeptide backbone. It has been found that their presence is essential for the antibody to mediate effector functions, such as ADCC (Lifely, M. R., et al., Glycobiology 5:813-822 (1995); Jefferis, R., et al., Immunol Rev. 163:59-76 (1998); Wright, A. and Morrison, S. L., supra).

Quantifiable properties of antibody isotypes and subclasses thought to confer in vivo activities such as ADCC, CA, and opsinization are shown below and described in e.g. Janeway et al. eds., 2001. Immunobiology 5: The immune system in health an disease, Garland Publishing, NY, N.Y., USA. Chapters 4 and 9. In one embodiment, the antibody or anti-target Ig or Mimetibody™ comprises an IgG heavy chain domain or defined fragment, for example, at least one of isotypes, IgG1, IgG2, IgG3 or IgG4, preferably an IgG1 class, e.g., as presented in Table 1 below (end of specification).

ADCC (antibody-dependent cellular cytotoxicity) activity and CDC (complement-directed cell lysis) activity, and Clq complement protein binding are cellular responses attributed to the consequences of cellular activation by binding of ab-ag complexes and by downstream sequelae caused by the release of cell mediators as a result of ab-ag complex binding to effector cells such as phagocytes, NK (natural killer cells), and mast cells. These cellular responses include neutralization of target, opsonization and sensitization (if antigen is displayed on the surface of a cell), sensitization of mast cells, and activation of complement.

Thus, the formation of a Fc-containing fusion protein which may be an antibody, an anti-target Ig, or a MIMETIBODY™, for the purposes of increasing circulating serum half-life, providing a multimerizing domain as is afforded by the dimerization of the chains at the hinge and the possibility to use the binding of Fc-domain to a Protein A affinity column are all important attributes of an Fc-fusion construct. Additional importance is derived from the cell and complement directed biological functions afforded by maintaining the structural integrity of the formed Fc structure.

An antibody or anti-target Ig or Mimetibody™ of the invention can include domains derived from any class (IgG, IgA, IgM, IgE, or IgD) or that contains an Fc receptor binding domain and thus has the desired spectrum of effector functions conferred by that isotype and subclass and can additionally comprise a kappa or lambda light chain.

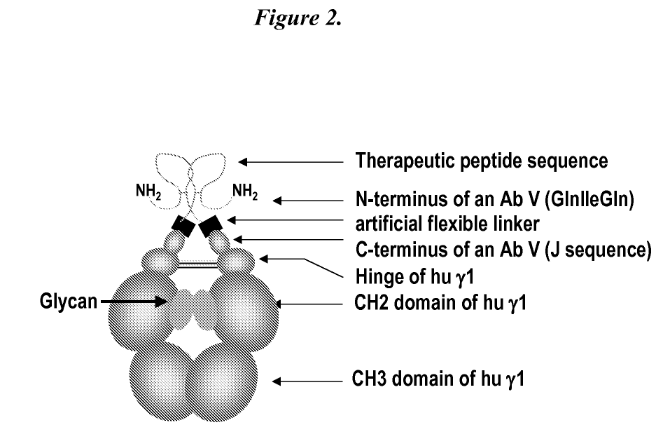

Applicants co-pending applications WO04/002417; WO04/002424; WO 05/081687; and WO05/032460 describe a structure referred to herein as a MIMETIBODY™ structure, each of which references are entirely incorporated herein by reference, and which structures are included in a ligand binding partners with immunoglobulin domains of the present invention, and which can include CDR-, CH1-deleted and/or hinge deleted mimetibodies as described in these and similar references and as otherwise known in the art. An non-limiting, exemplary Mimetibody™ is depicted in FIG. 2. The Mimetibody™ can comprise at least one CH3 region directly linked with at least one CH2 region directly linked with at least one hinge region or fragment thereof directly linked with an optional linker sequence, directly linked to at least one therapeutic peptide, optionally further directly linked with at least a portion of at least one variable antibody sequence. In a preferred embodiment, the Mimetibody™ comprises a pair of a bioactive peptide-linker-hinge-CH2-CH3 fusion polypeptides, the pair linked by association or covalent linkage, such as, but not limited to, a Cys-Cys disulfide bond. One example of such a composition comprises an EPO-mimetic peptide as the bioactive peptide. Thus, an EPO-mimetic CH1-deleted MIMETIBODY™ of the present invention mimics an antibody structure with its inherent properties and functions, while providing a therapeutic peptide and its inherent or acquired in vitro, in vivo or in situ properties or activities. The various portions of the antibody and therapeutic peptide portions of at least one EPO-Mimetibody™ of the present invention can vary as described herein in combination with what is known in the art.

In a typical embodiment an Fc-containing fusion protein or “Mimetibody™” comprises formula (I) which is absent the immunoglobulin CH1 domain:

V1ₒ-Pepₐ-Flexₙ-V2ₘ-Hinge-CH2-CH3 (I)

where Pep represents a bioactive peptide or polypeptide capable of specifically recognizing a target, Flex is an optional flexible linker polypeptide that provides structural flexibility by allowing the Mimetibody™ to have alternative orientations and binding properties, V1 and V2 are bracketing sequences, Hinge is at least a portion of an immunoglobulin hinge region, CH2 is at least a portion of an immunoglobulin CH2 constant region, CH3 is at least a portion of an immunoglobulin CH3 constant region; m,n and o can be zero or can be an integer between 1 and 10, and a can be an integer from 1 to 10. The Pep sequence can optionally include of sequences for the purposes of stabilization or any number of biophysical functions. In a typical embodiment, the bracketing sequences are derived from an antibody variable (V) domain such as a Vh framework and V1 is the sequence QIQ and V2 represents a sequence derived from an immunoglobulin J gene domain and is TLVTVSS (SEQ ID NO: 13). The resulting polypeptide can be linked to other polypeptides by association or covalent linkage, such as, but not limited to, a Cys-Cys disulfide bond.

Mimetibodies of the present invention thus provide at least one suitable property as compared to known proteins, such as, but not limited to, at least one of increased half-life, increased activity, more specific activity, a selected or more suitable subset of activities, less immunogenicity, increased quality or duration of at least one desired therapeutic effect, less side effects, and the like.

Fragments of mimetibodies according to Formula (I) can be produced by enzymatic cleavage, synthetic or recombinant techniques, as known in the art and/or as described herein. Mimetibodies can also be produced in a variety of truncated forms using antibody genes in which one or more stop codons have been introduced upstream of the natural stop site. The various portions of mimetibodies can be joined together chemically by conventional techniques, or can be prepared as a contiguous protein using genetic engineering techniques. For example, a nucleic acid encoding at least one of the constant regions of a human antibody chain can be expressed to produce a contiguous protein for use in mimetibodies of the present invention. See, e.g., Ladner et al., U.S. Pat. No. 4,946,778 and Bird, R. E. et al., Science, 242: 423-426 (1988), regarding single chain mimetibodies.

As used herein, the term “human antibody” or “human Mimetibody™” refers to an antibody in which substantially every part of the protein (e.g., target binding domain, framework, VL, VH, CH domains (e.g., CH2, CH3), hinge) is substantially non-immunogenic in humans with only minor sequence changes or variations. Such changes or variations optionally and preferably retain or reduce the immunogenicity in humans relative to non-modified human antibodies, or mimetibodies of the present invention. Thus, a human antibody, anti-target antibody or Mimetibody™ of the present invention is distinct from a chimeric or humanized antibody. It is pointed out that a human antibody, anti-target antibody or Mimetibody™ can be produced by a non-human animal or cell that is capable of expressing functionally rearranged human immunoglobulin (e.g., heavy chain) genes.

Human mimetibodies that are specific for at least one protein ligand or receptor thereof can be designed against an appropriate ligand, such as isolated and/or EPO protein receptor or ligand, or a portion thereof (including synthetic molecules, such as synthetic peptides). Preparation of such mimetibodies are performed using known techniques to identify and characterize ligand binding regions or sequences of at least one protein or portion thereof.

### Fc-Containing Protein Structures

Table 2 below (end of specification) gives exemplary Hinge sequences which are based on human IgG isotype sequences as defined in the literature (Dangl, J L et al. 1988. EMBO J. 7(7) 1989-1991). The pHinge sequence will comprise at least a hinge core sequence selected from terminal five residues of SEQ ID NO: 1-4 and, optionally, comprises a portion of the upper hinge sequences selected of SEQ ID NO: 1-4.

The hinge core, containing cysteine residues that link the two heavy chains via disulfide bonds, creates a molecule that is bivalent for antigen binding. The upper hinge is defined as the sequence between the end of CH1 to the first cysteine amino acid forming an inter-heavy chain disulfide bridge. Cysteine residues which are not part of the core hinge are involved in disulfide linkage to the light chain in a naturally occurring heterodimeric antibody. In a preferred embodiment, the fusion protein Hinge is the IgG1/2 hinge core (CPPCP) which is the 5 C-terminal residues of SEQ ID NO: 1 and 2. Sometimes called the lower hinge, is the N-terminal seven or eight residues of the CH2 region.

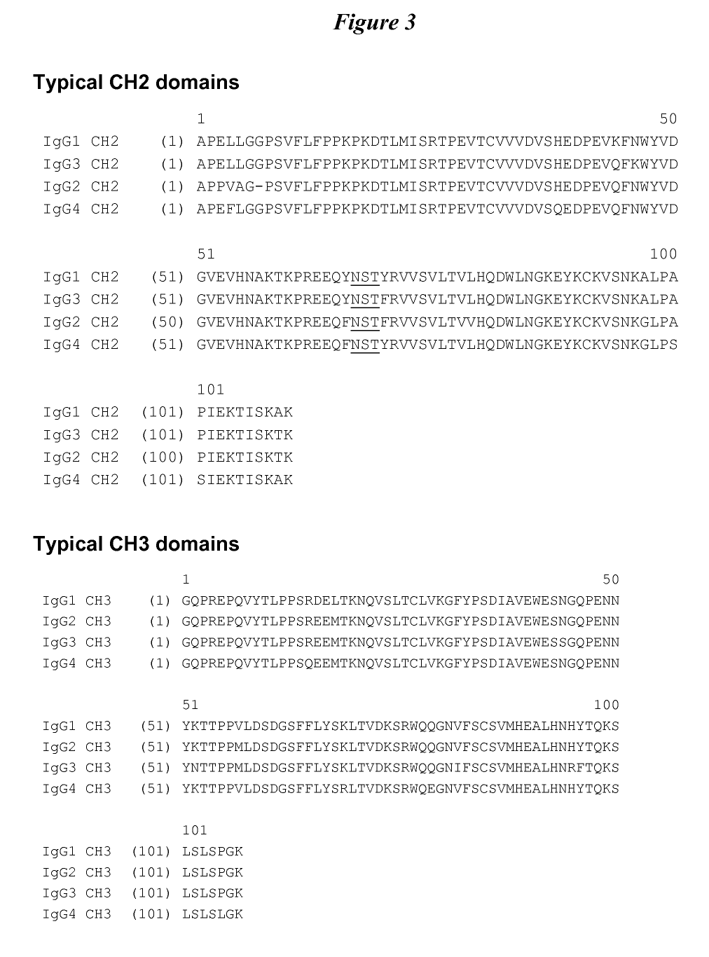

The CH2 domain of all IgG subtypes contains the unique N-glycosylation site of at residue 67 (FIG. 3, SEQ ID NOS: 5-8). The presence or absence of glycan in the Fc-containing molecule affects the affinity for one or more of the FcγRI, FcγRIIA, and FcγRIIIA receptors, ADCC activity, macrophage or monocyte activation, and serum half-life (Lifely et al., Jeffreis, and Wright and Morrison, supra). Recombinant production of antibodies and Mimetibody™ constructs by eukaryotic cells will affect the decoration of final composition with a glycan structure typical of the host cell and which glycan structure may be further influenced by the cell culture conditions.

With respect to the tertiary structure affected by the hinge core interchain disulfide bonds along with the attached glycan, the Fc-containing proteins represent a unique challenge for PEGylation because non-antigen binding properties of antibody reside in the Fc. Thus, a conjugation method that does not significantly impact the glycan structure or the ability of the heavy chain polypeptides to form interchain disulfide bonds is important in the PEGylation of Fc-containing proteins in order to retain biologic activity of the final composition in vivo.

The constant region of antibody refers to a region other than the variable region proposed by Kabat et al. (Kabat, “Sequence of Proteins of Immunological Interest,” U.S. Department of Health and Human Services (1983)). The Fc moiety refers to a region which is not involved in the binding with the antigen and which is primarily responsible for the effector function among the fragments cleaved with a proteolytic enzyme, papain.

In one aspect, the Fc-containing protein of the invention is formed through the complexing (multimerizing) of Fc polypeptide sequences. By Fc polypeptide sequences is meant domains that typically comprise an Fc as defined above. The polypeptides of the dimeric structure may or may not contain the same sequences and/or domains, provided they are capable of dimerizing to form an Fc region (as defined herein). A Fc polypeptide is generally contiguously linked to one or more domains of an immunoglobulin heavy chain or other suitable bioactive peptide in a single polypeptide, as described and shown in Formula I, for example. In one embodiment, the Fc polypeptide comprises at least a portion of a hinge sequence, at least a portion of a CH2 domain and/or at least a portion of a CH3 domain. In one embodiment, the antibody constant domain is a CH2 and/or CH3 domain. In any of the embodiments of an antibody fragment of the invention that comprises a constant domain, the antibody constant domain can be from any immunoglobulin class, for example an IgG. The immunoglobulin source can be of any suitable species of origin (e.g., an IgG may be human IgG₁) or of synthetic form. Suitable CH2 and CH3 domains are those CH2 domains provided as SEQ ID NOS: 5-8 and suitable CH3 domains are provided as SEQ ID NOS: 9-12. Other suitable hinge, CH2 and CH3 domains are given in applicants co-pending applications, PCT WO05/005604 and U.S. Ser. No. 10/872,932 each entirely incorporated herein by reference. It will be readily apparent to one skilled in the art that modification, such as truncations, insertions and deletions; and substitutions, such as conservative amino acid substitutions; of the sequences provided can also be made while maintaining the desired Fc functions imparted by the primary, secondary, tertiary and higher order Fc-structures. A conservative amino acid substitution refers to the replacement of a first amino acid by a second amino acid that has chemical and/or physical properties (e.g, charge, structure, polarity, hydrophobicity/hydrophilicity) that are similar to those of the first amino acid. Conservative substitutions include replacement of one amino acid by another within the following groups: lysine (K), arginine (R) and histidine (H); aspartate (D) and glutamate (E); asparagine (N), glutamine (Q), serine (S), threonine (T), tyrosine (Y), K, R, H, D and E; alanine (A), valine (V), leucine (L), isoleucine (I), proline (P), phenylalanine (F), tryptophan (W), methionine (M), cysteine (C) and glycine (G); F, W and Y; C, S and T.

### Modification of Fc-Containing Proteins

The chemical modification of proteins in order to gain longer in vivo circulation time has typically relied upon methods of addition of one or more PEG moieties of several thousand Daltons. Other hydrophilic polymers may also be used and examples are given below. A variety of methods are available for linking PEG and other substituents to proteins. Very often the epsilon-amine of the lysine sidechain or the single alpha amino group at the N-terminus are used as the site of modification due the reactive nature of a primary amine (Zalipsky and Lee. 1992. In: Poly(ethylene glycol) Chemistry: Biotechnical and Biomedical Applications, J. M. Harris, ed. Plenum, New York, N.Y., pp. 347-370). Amine-reactive groups include electrophilic groups such as tosylate, mesylate, halo (chloro, bromo, iodo), N-hydroxysuccinimidyl esters (NHS), substituted phenyl esters, acyl halides, and the like.

Linkage to free thiol groups in the protein is another option and thiol reactive reagents include: maleimide, iodoacetyl, acrylolyl, pyridyl disulfides, 5-thiol-2-nitrobenzoic acid thiol (TNB-thiol), and the like. However, thiol groups of cysteine are integral to the structural integrity of the protein as disulfide linkages and therefore sometimes unavailable for conjugation.

Urethane (carbamate) attachment of PEG to a protein is another convenient way to form PEG-protein conjugates and carbamate linkages are more resistant to hydrolysis as compared to amide linkages. There are a few known PEG reagents that are used to make urethane linked PEG-proteins, such as slow-reacting imidazolyl formate, trichlorophenyl carbonate, and nitrophenyl carbonates (NPC) derivatives. A more reactive reagent, mPEG-succinimidyl carbonate (mPEG-SC), can also be utilized.

PEGylation of proteins with slow-reacting reagents such as PEG-NPC (PEG-bis-(4-nitrophenyl)-carbonate) proceeds more efficiently at a pH range of 8-10, as most amino groups of proteins are deprotonated and behave as nucleophiles (Bronsted bases) at this pH. PEG-NPC is not very reactive at pH below 8, thus, these reactions proceed very slowly and not efficiently.

An alternative method of generating PEGylated peptides and proteins is provided herein which allows for efficient modification of Fc-containing proteins at pH at or below 7. The method is also useful to optimize the yield of lightly PEGylated Fc-containing proteins, wherein “lightly” PEGylated refers to a ratio of PEG to protein of 1:1, 2:1, or 3:1. The method is also useful for PEGylating Fc-containing proteins that are poorly soluble at pH at or below 7.

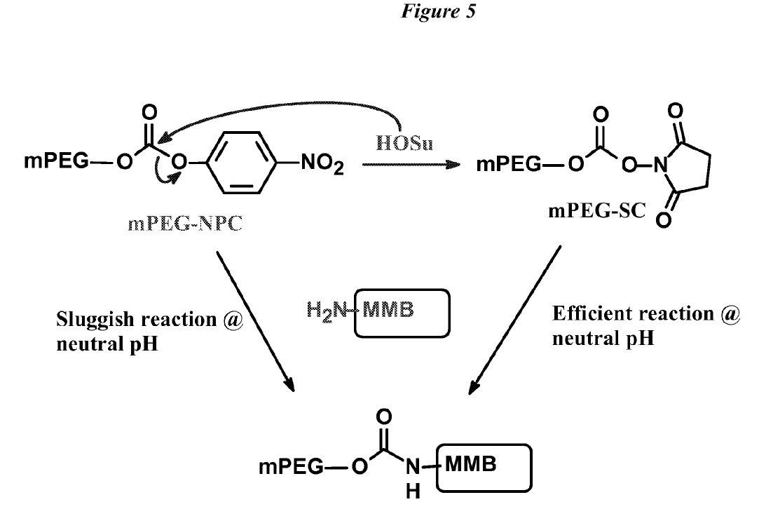

FIG. 5 shows a reaction scheme for modification of Fc-containing proteins, such as a Mimetibody™. The reaction involves the use an acylating derivative like mPEG-NPC for formation of amide- or urethane-linked PEG proteins. Included in the reaction is an activating agent effective to increase the reaction efficiency and allow facile protein modification at or below pH 7.0 conditions. As seen in FIG. 5, the activating agent, exemplified in the reaction scheme as n-hydroxysuccinimide (HOSu), readily reacts with the acylating derivative mPEG-NPC, to form mPEG-hydroxysuccinimide (mPEG-SC). mPEG-SC reacts with the protein, designated in FIG. 5 as a Mimetibody™ bearing a reactive amine, H₂N-MMB.

The activating agent is preferably a water-soluble, non-carboxylic, Bronsted acid of moderate acidity having the propensity to donate N- or O-linked protons to the PEGylation reagent. General examples include acidic alcohols, phenols, imidazols, triazols, and tetrazols, among others. Examples of acidic acids suitable for use in the reaction are set forth in co-pending application No. 60/686,738, filed Jun. 1, 2005, which is incorporated by reference herein. Briefly, exemplary activating agents include, but are not limited to, N-hydroxydicarboxylmides, N-hydroxyphthalimides particularly with nitro and other electron withdrawing substituents on the aromatic ring, N-hydroxy tetrahydrophthalimide, N-hydroxyglutarimide, N-hydroxy-5-norbornene-2,3-dicarboxylmide, and N-hydroxy-7-oxabicyclo[2.21]hept-5-ene-2,3-dicarboxylmide. 1-N-hydroxybenzotriazol and derivatives with electron withdrawing groups on the aromatic ring, e.g. nitro, chloro, 3-hydroxy-1,2,3-benzotriazin-4(3H)-one. N-hydroxysulfosuccinimide sodium salt is very soluble in water, which means that it can be used at even higher concentration in aqueous buffers than HOSu. Exemplary hydroxy amine derivatives, in addition to N-hydroxysuccinimide (HOSu), include sulfonate derivatives of HOSu, 1-hydroxybenzotriazole (HOBt), and hydroxyl-7-azabenzotriazole (HOAt). These coupling agents can act as an efficient buffer component in a pH range of about 4 to about 7.5, based on their pKa. For example, HOSu, being a weak acid of pKa=6, acts as an efficient buffer component in a pH range of about 5 to about 7. The coupling reagent may be added to a buffer, or may comprise the buffer with or without other salts. Further, as HOSu is quite soluble in an aqueous solution, it can be added to buffers at relatively high concentration to further boost the PEGylation reaction.

Exemplary phenols include, but are not limited to, dinitrophenol, trinitrophenol, trifluorophenol, pentafluorophenol, and pentachlorophenol. In addition, 4- or 2-hydroxypyridine and derivatives are also suitable for use, as exemplified by hydroxyl-2-nitropyridine.

Other exemplary compounds for use as the activating agent include compounds having an acidic N—H functionality, such as imidazol derivatives with electron withdrawing groups (imidazol, pKa=7), e.g. 4- or 2-nitroimidazol, triazol, tetrazol, and some derivatives, such as 2-nitro-1,2,4-triazole.

The method is particularly suited for use with acylating PEG reagents of low to medium reactivity, particularly reagents having low to medium reactivity at room temperature and/or at pH≦7.0. A particularly preferred acylating reagent is mPEG-NPC. Other low-reactivity PEG reagents suitable for use in the present invention include, but are not limited to, carbonyl imidazolyls, trichlorophenyl carbonates, and other nitrophenyl carbonates, which have been utilized to make urethane-linked PEG-proteins.

Methoxy-PEG (mPEG) is exemplified herein, yet other modified PEGs can be used. In one embodiment, a monofunctional PEG having an inert group modification to one end of the polymer is used. Examples include, but are not limited to, short alkoxy PEG derivatives (ethoxy, butoxy, and the like) or PEG modified with various protected functional groups, as would be understood by one of skill in the art.

It will further be appreciated that while a PEG reagent having a molecular weight of 30,000 Da is exemplified herein, other PEG lengths are also contemplated for use in the present method. A preferred size range of PEG is 1,000-50,000 Da. Preferably, the PEG length is 40,000 Da or less, more preferably 30,000 Da or less.

It will also be appreciated that PEG is merely exemplary of a variety of hydrophilic polymers that are suitable for modification of proteins. In general, a hydrophilic polymer is one having moieties soluble in water and that lend to the polymer some degree of water solubility at room temperature. Exemplary hydrophilic polymers include polyvinylpyrrolidone, polyvinylmethylether, polymethyloxazoline, polyethyloxazoline, polyhydroxypropylyoxazoline, polyhydroxypropyl-methacrylamide, polymethacrylamide, polydimethyl-acrylamide, polyhydroxypropylmethacrylate, polyhydroxyethylacrylate, hydroxymethylcellulose, hydroxyethylcellulose, polyaspartamide, copolymers of the above-recited polymers, and polyethyleneoxide-polypropylene oxide copolymers. Properties and reactions with many of these polymers are described in U.S. Pat. Nos. 5,395,619 and 5,631,018.

In one embodiment, the method preferably includes the step of combining the protein with an activating agent and mPEG-NPC in an aqueous medium at a pH of less than about 8.0. In one preferred embodiment, the pH is about 5.0 or 6.0 to about 7.5. In other embodiments, the pH is less than about 7 or 8. It will be appreciated that neutral conditions are often more favorable for protein stability and/or solubility. Neutral conditions also generally favor modification of the most reactive and least basic amino groups on a protein. As the N-terminal amino group is usually a few orders of magnitude less basic (pKa=7.9) than the ε-amino group of lysine (pKa=10-11), lower pH tends to keep most of the latter groups fully protonated. Therefore, a lower pH is generally more favorable for selective modification of the N-terminal amino group. It will be appreciated that often proteins PEGylated predominantly on the N-terminal amino retain higher functional activity.

The protein, activating agent, and mPEG-NPC are combined for a period of time from about 0.5 hours to about 24 hours. In one embodiment, the time is from about 2 hours to about 6 hours. It will be appreciated that one of skill in the art can readily determine and/or vary the time to optimize the reaction.

The reaction temperature is generally about room temperature, or between 8° C. and 37° C., however, it will be appreciated that one of skill in the art can readily determine and/or vary the temperature to optimize the reaction.

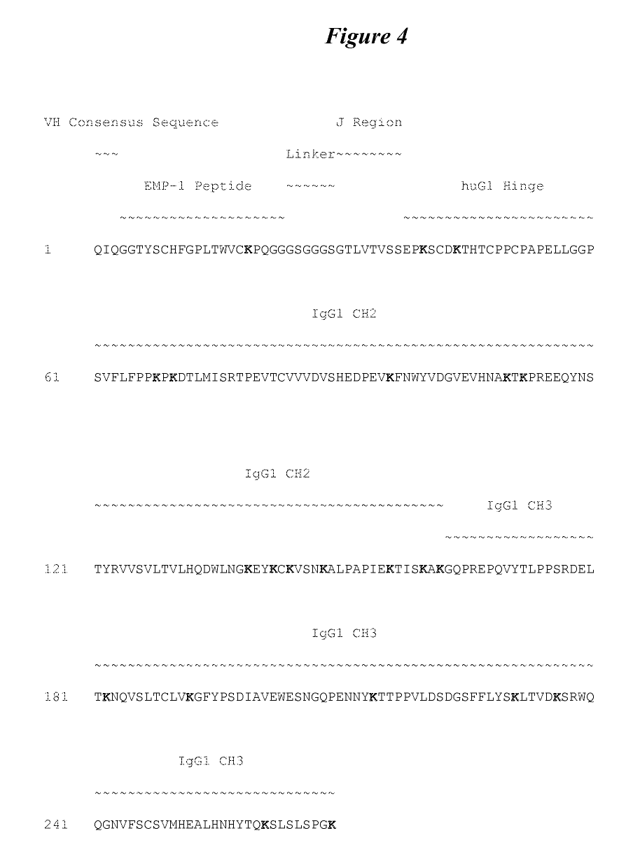

The applicants have exemplified the invention by making various batches of conjugated EPO-mimitibody (CNTO528, SEQ ID NO: 14, FIG. 4) which is a homodimer of a fusion protein comprising a IgG1 hinge region, as well as CH2 and CH3 regions of a human IgG1 molecule and thus comprises an immunoglobulin Fc. The methods used to conjugate hydrophilic polymer (PEG) according to the reaction scheme of FIG. 5 were used as described in Examples 1 and 2. The products of these reactions, which have been characterized using biophysical and physiochemical measurements can be further purified and pooled in order to give a more or less heterogeneous preparation. In a typical embodiment, the preparation the predominant species has a ratio of 2:1 PEG:polypeptide. Bioassay of the preparations (Examples 3 and 4) indicated that functional activity as measured by the ability of the conjugate to stimulate hematopoietic cell proliferation similar to the unconjugated material was retained. Moreover, conjugate so prepared was found to exhibit desirable pharmacokinetic and pharmacodymamic properties as compared to unconjugated material as evidenced by an increase in mean serum residence time and increase in total exposure. The preparations were further shown to cause a longer and more pronounced increase in blood parameters indicative of increased red cell and hemoglobin production.

### Biological Characterization

Generally, Fc-containing proteins can be compared for functionality by several well-known in vitro assays. In particular, affinity for members of the FcγRI, FcγRII, and FcγRIII family of Fcγ receptors is of interest. These measurements could be made using recombinant soluble forms of the receptors or cell-associated forms of the receptors. In addition, affinity for FcRn, the receptor responsible for the prolonged circulating half-life of IgGs, can be measured, for example, by BIAcore using recombinant soluble FcRn. Cell-based functional assays, such as ADCC assays and CDC assays, provide insights into the likely functional consequences of particular variant structures. In one embodiment, the ADCC assay is configured so as to have NK cells be the primary effector cell, thereby reflecting the functional effects on the FcγRIIIA receptor. Phagocytosis assays may also be used to compare immune effector functions of different variants, as can assays that measure cellular responses, such as superoxide or inflammatory mediator release. In vivo models can be used as well, as, for example, in the case of using variants of anti-CD3 antibodies to measure T cell activation in mice, an activity that is dependent on Fc domains engaging specific ligands, such as Fcγ receptors.

### Protein Production Processes

In a preferred embodiment, at least one the CH1-deleted Mimetibody™ or specified portion or variant of the present invention is produced by at least one cell line, mixed cell line, immortalized cell or clonal population of immortalized and/or cultured cells. Immortalized protein producing cells can be produced using suitable methods. Preferably, at least the CH1 deleted Mimetibody™ or specified portion or variant is generated by providing nucleic acid or vectors comprising DNA derived or having a substantially similar sequence to, at least one human immunoglobulin locus that is functionally rearranged, or which can undergo functional rearrangement, and which further comprises a Mimetibody™ structure as described herein, e.g., but not limited to Formula (I), wherein portions of C- and N-terminal variable regions can be used for V1 and V2, hinge regions for pHinge, CH2 for CH2 and CH3 for CH3, as known in the art.

The host cells described herein comprise host cells capable of producing specific antibodies which are glycosylated. The host cell can optionally be selected from COS-1, COS-7, HEK293, BHK21, CHO, BSC-1, Hep G2, 653, SP2/0, 293, HeLa, myeloma, lymphoma, yeast, insect or plant cells, or any derivative, immortalized or transformed cell thereof. Also described is a method for producing an antibody or Fc-fusion protein, comprising translating the encoding nucleic acid under conditions in vitro, in vivo or in situ, such that the peptide or antibody is expressed in detectable or recoverable amounts.

Different processes involved with the production of Fc-containing proteins can impact Fc oligosaccharide structure. In one instance, the host cells secreting the Fc-containing protein are cultured in the presence of serum, e.g., FBS, that was not previously subjected to an elevated heat treatment (for example, 56° C. for 30 minutes). This can result in Fc-containing protein that contains no, or very low amounts of, sialic acid, due to the natural presence in the serum of active sialidase enzymes that can remove sialic acid from the Fc-containing proteins secreted from those cells. In another embodiment, the cells secreting the Fc-containing protein are cultured either in the presence of serum that was subjected to an elevated heat treatment, thereby inactivating sialidase enzymes, or in the absence of serum or other medium components that may contain sialidase enzymes, such that the Fc-containing protein has higher or lower levels of glycosylation.

In another embodiment, the conditions used to purify and further process Fc-containing proteins are established that will favor optimal glycan content. In one embodiment, the conditions produce maximal or minimal oligosaccharide content or cause the transformation of the expressed Fc-containing polypeptide in a predominant glycoform. For example, because sialic acid is acid-labile, prolonged exposure to a low pH environment, such as following elution from Protein A chromatography column or viral inactivation efforts, may lead to a reduction in sialic acid content.

In another embodiment, the Fc-containing fusion protein of the invention is aglycosylated. Thus, the host cell may be selected from a species or organism incapable of glycosylating polypeptides, e.g. a prokaryotic cell or organism, such as and of the natural or engineered E. coli spp, Klebsiella spp., or Pseudomonas spp. Alternatively, the protein may be produced in a eukaryotic host cell but subjected to enzymatic deglycosylation using, e.g. peptide N-glycosidase F.

### Host Cell Selection or Host Cell Engineering

As described herein, the host cell chosen for expression of the recombinant Fc-containing protein or monoclonal antibody is an important contributor to the final composition, including, without limitation, the variation in composition of the oligosaccharide moieties decorating the protein in the immunoglobulin CH2 domain. Thus, one aspect of the invention involves the selection of appropriate host cells for use and/or development of a production cell expressing the desired therapeutic protein.

In one embodiment, the host cell may be naturally devoid of, or is genetically modified to be devoid of, glycosyltransferases, such that antibodies expressed in said cells lack ability to add a specific sugar residue, e.g. sialic acid, to the glycan. In a separate embodiment, the host cell cell may naturally overexpress, or be genetically modified to overexpress, a glycosylase enzyme that removes a specific sugar residue from the glycan attached to the antibody during production, e.g galactase. Such a glycosylase enzyme may act intracellularly on antibodies before the antibodies are secreted or be secreted into the culture medium and act on antibodies that have already been secreted into the medium and may further contain, e.g. galactase. Methods of selecting cell lines with altered glycosylases and which express glycoproteins with altered carbohydrate compositions have been described (Ripka and Stanley, 1986. Somatic Cell Mol Gen 12:51-62; US2004/0132140). Methods of engineering host cells to produce antibodies with altered glycosylation patterns resulting in enhanced ADCC have been taught in, e.g., U.S. Pat. No. 6,602,864, wherein the host cells harbor a nucleic acid encoding at least one glycoprotein modifying glycosyl transferase, specifically β (1,4)—N-acetylglucosaminyltranferase III (GnTIII).

Other approaches to genetically engineering the glycosylation properties of a host cell through manipulation of the host cell glycosyltransferase involve eliminating or suppressing the activity, as taught in EP1,176,195, specifically, alpha-1,6 fucosyltransferase (FUT8 gene product). It would be obvious to one skilled in the art to practice the methods of host cell engineering in other than the specific examples cited above. Further, the engineered host cell may be of mammalian origin or may be selected from COS-1, COS-7, HEK293, BHK21, CHO, BSC-1, Hep G2, 653, SP2/0, 293, HeLa, myeloma, lymphoma, yeast, insect or plant cells, or any derivative, immortalized or transformed cell thereof.

### Ligand Binding Peptide Fragments Comprising the Fusion Protein

The ligand binding peptide comprising the Mimetibody™ or antibody fusion protsin of the present invention may be one or more known peptides, either agonist or antagonist or ligand binding. Such ligand binding peptides can be known peptides such as naturally occurring or isolated proteins or any portion thereof that binds a ligand or receptor, or may be a portion of a receptor or other protein. Synthetic peptides can also be used that bind to a desired ligand. Non-limiting examples of such peptides are well known in the art. Non-limiting examples of such ligand binding peptides are disclosed in PCT WO05081 687A2, published 9 Sep. 2005; WO05032460A2, published 14 Apr. 2005; WO04002417A2, published 8 Jan. 2004; and WO03084477A2, published 16 Oct. 2003, each of which is entirely incorporated herein by reference.

### Antibody Fragments Comprising the Fusion Protein

An antibody constant domain described in this application can include or be derived from any mammal, such as, but not limited to, a human, a mouse, a rabbit, a rat, a rodent, a primate, or any combination thereof and includes isolated human, primate, rodent, mammalian, chimeric, humanized and/or CDR-grafted antibodies, immunoglobulins, cleavage products and other specified portions and variants thereof.

The invention also relates to Fc-containing fusion protein encoding or complementary nucleic acids, vectors, host cells, compositions, formulations, devices, transgenic animals, transgenic plants, and methods of making and using thereof, as described herein together as combined with what is known in the art.

The antibodies or Fc-fusion proteins described herein can be derived in several ways well known in the art. In one aspect, antibody fragments are conveniently obtained from antibody encoding nucleic acids from hybridomas prepared by immunizing a mouse with the target peptides. The antibodies can thus be obtained using any of the hybridoma techniques well known in the art, see, e.g., Ausubel, et al., ed., Current Protocols in Molecular Biology, John Wiley & Sons, Inc., NY, N.Y. (1987-2006); Sambrook, et al., Molecular Cloning: A Laboratory Manual, 2^{nd} Edition, Cold Spring Harbor, N.Y. (1989); Harlow and Lane, antibodies, a Laboratory Manual, Cold Spring Harbor, N.Y. (1989); Colligan, et al., eds., Current Protocols in Immunology, John Wiley & Sons, Inc., NY (1994-2006); Colligan et al., Current Protocols in Protein Science, John Wiley & Sons, NY, N.Y., (1997-2006), each entirely incorporated herein by reference.

The antibodies or Fc-fusion proteins or components and domains thereof may also be obtained from selecting from libraries of such domains or components, e.g., a phage library. A phage library can be created by inserting a library of random oligonucleotides or a library of polynucleotides containing sequences of interest, such as from the B-cells of an immunized animal or human (Smith, G. P. 1985. Science 228: 1315-1317). Antibody phage libraries contain heavy (H) and light (L) chain variable region pairs in one phage allowing the expression of single-chain Fv fragments or Fab fragments (Hoogenboom, et al. 2000, Immunol. Today 21(8) 371-8). The diversity of a phagemid library can be manipulated to increase and/or alter the immunospecificities of the monoclonal antibodies of the library to produce and subsequently identify additional, desirable, human monoclonal antibodies. For example, the heavy (H) chain and light (L) chain immunoglobulin molecule encoding genes can be randomly mixed (shuffled) to create new HL pairs in an assembled immunoglobulin molecule. Additionally, either or both the H and L chain encoding genes can be mutagenized in a complementarity determining region (CDR) of the variable region of the immunoglobulin polypeptide, and subsequently screened for desirable affinity and neutralization capabilities. Antibody libraries also can be created synthetically by selecting one or more human framework sequences and introducing collections of CDR cassettes derived from human antibody repertoires or through designed variation (Kretzschmar and von Ruden 2000, Current Opinion in Biotechnology, 13:598-602). The positions of diversity are not limited to CDRs, but can also include the framework segments of the variable regions or may include other than antibody variable regions, such as peptides.

Other libraries of target binding components which may include other than antibody variable regions are ribosome display, yeast display, and bacterial displays. Ribosome display is a method of translating mRNAs into their cognate proteins while keeping the protein attached to the RNA. The nucleic acid coding sequence is recovered by RT-PCR (Mattheakis, L. C. et al. 1994. Proc. Natl. Acad. Sci. USA 91, 9022). Yeast display is based on the construction of fusion proteins of the membrane-associated alpha-agglutinin yeast adhesion receptor, aga1 and aga2, a part of the mating type system (Broder, et al. 1997. Nature Biotechnology, 15:553-7). Bacterial display is based fusion of the target to exported bacterial proteins that associate with the cell membrane or cell wall (Chen and Georgiou 2002. Biotechnol Bioeng, 79:496-503).

In comparison to hybridoma technology, phage and other antibody display methods afford the opportunity to manipulate selection against the antigen target in vitro and without the limitation of the possibility of host effects on the antigen or vice versa.

Also described is a method for producing an antibody or Fc-fusion protein, comprising translating the encoding nucleic acid under conditions in vitro, in vivo or in situ, such that the peptide or antibody is expressed in detectable or recoverable amounts.

While having described the invention in general terms, the embodiments of the invention will be further disclosed in the following examples.

### EXAMPLE 1

### Preparation of PEG_{30K}CNTO528

CNTO528, an Epo receptor agonist as described WO04/002417, was selected as a model biomolecule (Mimetibody™). In CNTO528, the sequence of an Epo mimetic peptide (EMP-1) known to require dimerization for bioactivity is fused to the hinge and Fc portion of IgG1, resulting in an active Epo receptor agonist. There are 21 lysine residues in the Fc and hinge portion of CNTO528 and the Epo mimetic peptide has one lysine residue (FIG. 4, SEQ ID NO: 14). In addition, there are two amino terminal groups on a single Mimetibody™ molecule (46 total potential sites). Although the Fc portion contributes to a longer circulation time compared to the free peptide, even longer circulation may be desired for improved dosing regimens.

Amine-directed PEGylation of CNTO528 was performed as follows, and according to the reaction scheme illustrated in FIG. 5. Nitrophenyl carbonate derivatized methoxy-polyethylene glycol, 30,000 Daltons, (mPEG₃₀ₖ-NPC) was obtained from NOF Corporation (Tokyo, Japan, lot #M35525). A 10 mM solution of PEG₃₀ₖ-NPC in acetonitrile was prepared just prior to use. The Mimetibody™ CNTO528 was prepared as described in WO04/002417; WO04/002424; WO 05/081687; and WO05/032460. A buffer of 100 mM HEPES and 100 mM N-hydroxysuccinimide (HOSu), pH 7.5 was prepared.

PEG₃₀ₖ-NPC was used in 10-fold molar excess to CNTO528. PEG solution was added to CNTO528 in buffer and water to a final protein concentration of 4 mg/mL, a final buffer concentration of 25 mM HEPES/HOSu, and a final PEG concentration of 0.645 mM. The reaction was allowed to proceed at room temperature (21-22° C.) in the dark on a rocking mixer for 4 hours and then placed at 4° C. overnight. The reaction was stopped with 30 mM final concentration of glycine.

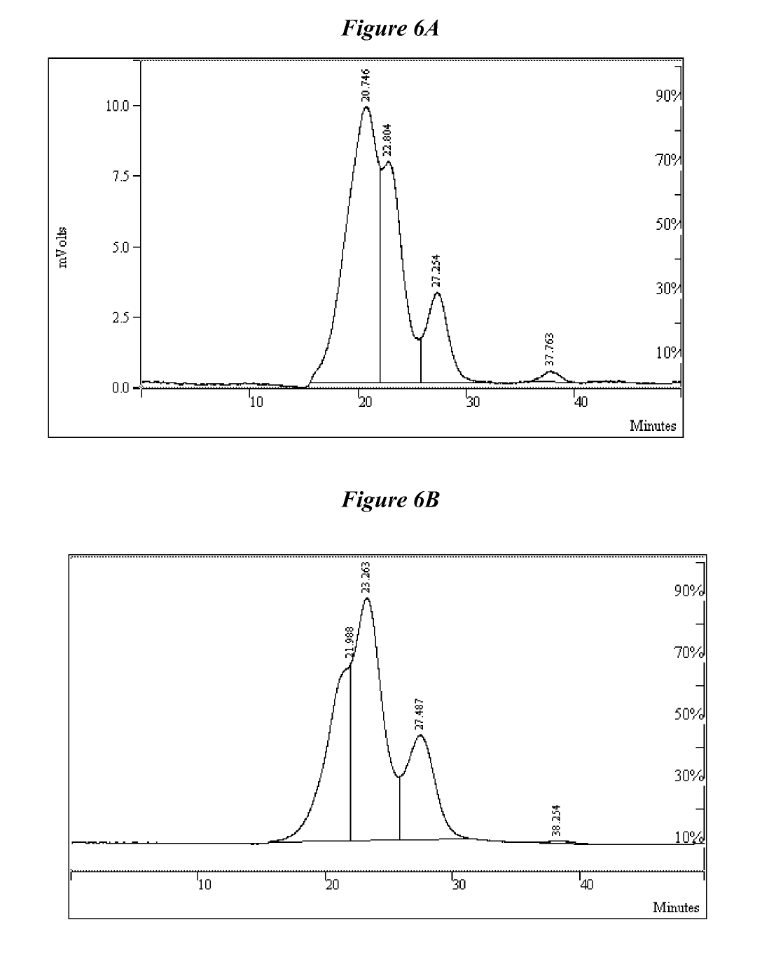

The crude reaction material was dialyzed in 10 mM citrate, pH 5.0, and then purified by cation exchange chromatography (SP HP 1 mL or 5 mL column, Amersham Biosciences) using a NaCl elution gradient. The reaction material was characterized by size-exclusion chromatography (SEC) using Superose 6 (Amersham Biosciences) in a 50 mM sodium phosphate and 100 mM NaCl, pH 7.4 mobile phase. Following analysis by SEC, fractions were pooled to obtain the desired species ratio. The SEC chromatogram for crude material is shown in FIG. 6A and chromatogram details are summarized in Table 3 below (end of specification).

The crude mixture was predominantly composed of 3:1 PEG-protein, with 55% of the conjugates having an average of three PEG chains per Mimetibody™. The remainder of the composition was 31% 2:1 PEG-protein and 12.5% 1:1 PEG-protein, with a small amount of free Mimetibody™ protein present.

Next, the fractions were pooled to compose a predominately 2:1 PEG protein mixture and dialyzed into phosphate buffered saline (PBS), pH 7.2, and filtered using a 0.2 μm pore-size membrane to sterilize. PEG_{30K}-CNTO528 was placed in a sterile glass vial at 2 mL±0.1 mL, as determined by A280 (1.0 mg/mL). This conjugate mixture was characterized by SEC as above and the results are shown in Table 4 below (end of specification) and in FIG. 6B.

After purification and pooling of fractions to enhance the amount of 2:1 conjugate in the mixture, the composition was predominantly (48%) 2:1 PEG-protein conjugate. The remainder of the composition was 30% 2:1 PEG-protein and 21.4% 1:1 PEG-protein, with a small amount of free Mimetibody™ protein present.

### Example 2

### Preparation of PEG₃₀ₖCNTO528

A second reaction was done, where the initial starting amounts of certain reactants as described in Example 1 was varied. Conjugate was prepared, with the following changes to the amounts of the reaction components: the final concentrations for CNTO528, HEPES/HOSu buffer, and PEG₃₀ₖ-NPC were 5.6 mg/mL, 35 mM, and 0.9 mM respectively. The reaction was stopped glycine at a final concentration of 40 mM.

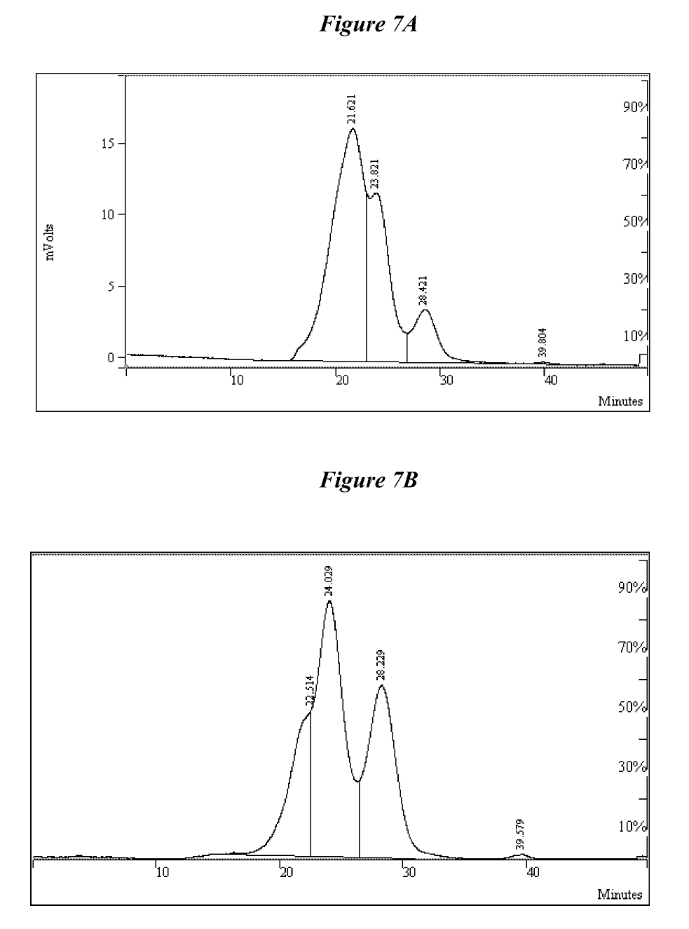

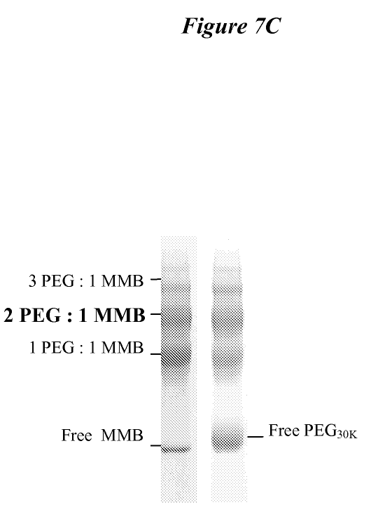

The conjugate was characterized by size-exclusion chromatography (SEC) using Superose 6 (Amersham Biosciences) in a 50 mM sodium phosphate and 150 mM NaCl, pH 7.0 mobile phase and the results for crude material is shown in FIG. 7A with chromatogram details in the Tables 5 below (end of specification)

After purification and pooling to increase the amount of 1:1 and 2:1 PEG-protein conjugates, the mixture was again analyzed by SEC and the results are shown in FIG. 7B with the chromatographic analysis given in Table 6 below (end of specification).

FIG. 7A shows that in the mixture after reaction, the 3:1 PEG-Mimetibody™ conjugate (peak at 21.6 minutes) made up 60% of the composition. The 2:1 and 1:1 PEG-Mimetibody™ conjugates comprised 29% and 10% of the mixture. The peak with an elution time of 22.5 minutes in FIG. 7B corresponds to a 3:1 PEG-protein conjugate and is about 20% of the total. The peaks that elute at 24 minutes and 28.2 minutes correspond to 2:1 and 1:1 PEG-protein conjugates, respectively. The small peak observed at 39.6 minutes corresponds to free protein (CNTO528). From the area of each peak, the percentage of each conjugate was calculated, and found to be 20% 3:1, 46.2% 2:1, 32.7% 1:1, and 1.05% free protein.

The conjugate was also analyzed by gel electrophoresis under denaturing conditions, using NuPAGE® Bis-Tris 4-12% gradient gel and MOPS-SDS running buffer (Invitrogen Life Technology, Carlsbad, Calif.). Gels were loaded with 6 μg per lane and run at a constant voltage of 200 volts for 55 minutes. One gel was first stained in iodine for PEG detection and then after evaporating the iodine, in Coomassie Blue for protein detection. Exemplary lanes from each gel are shown in FIG. 7C, where the left lane shows the protein stain and the right lane shows the PEG stain. The 3:1, 2:1, and 1:1 PEG-CNTO528 conjugates are visible, with the darkest Coomassie stained band for the 2:1 conjugate confirming that this conjugate is present as a significant fraction of the total.

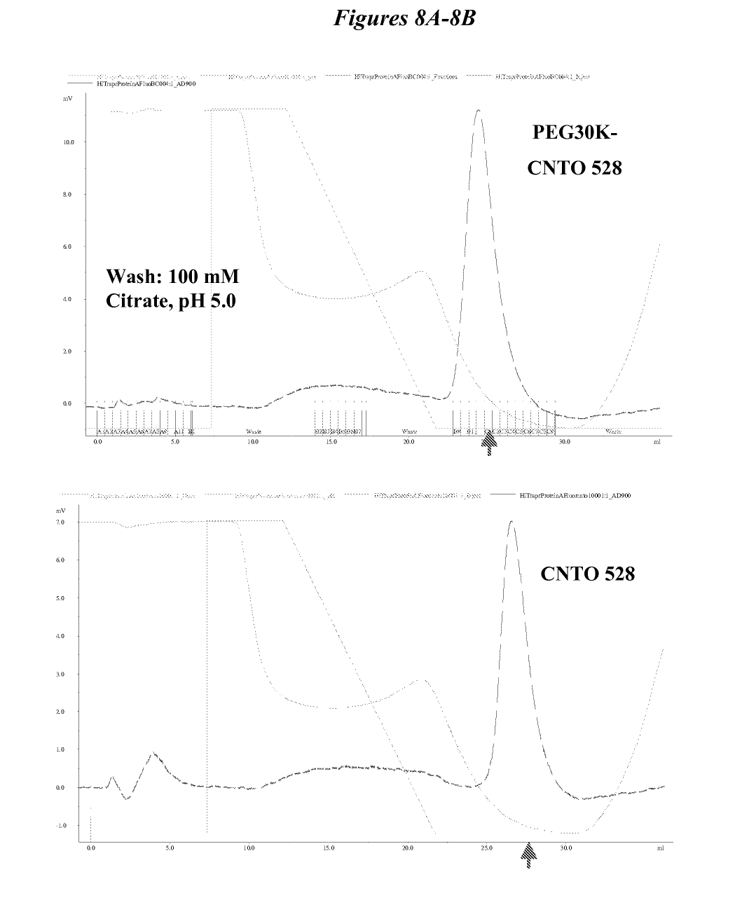

The conjugate was also characterized by high performance liquid chromatography using a Protein A column. Samples were injected onto a 1 mL Hi Trap Protein A FF column (GE Healthcare) in 50 mM NaPO₄/150 mM NaCl, pH 7.4 buffer. After a 5 column volume wash in 100 mM citrate, pH 5.0, a gradient to 100 mM Citrate, pH 3.5 eluted the samples. The HPLC trace for the conjugate and for the conjugate PEG₃₀ₖ-CNTO528 (FIG. 8A) and of the unmodified Mimetibody™, CNTO528, on a Protein A column (FIG. 8B). The unmodified CNTO528 binds to Protein A and elutes at about 26 minutes. The PEGylated CNTO528 retains binding to Protein A, with a slight shift in the peak time to about 25 minutes (FIG. 8A).

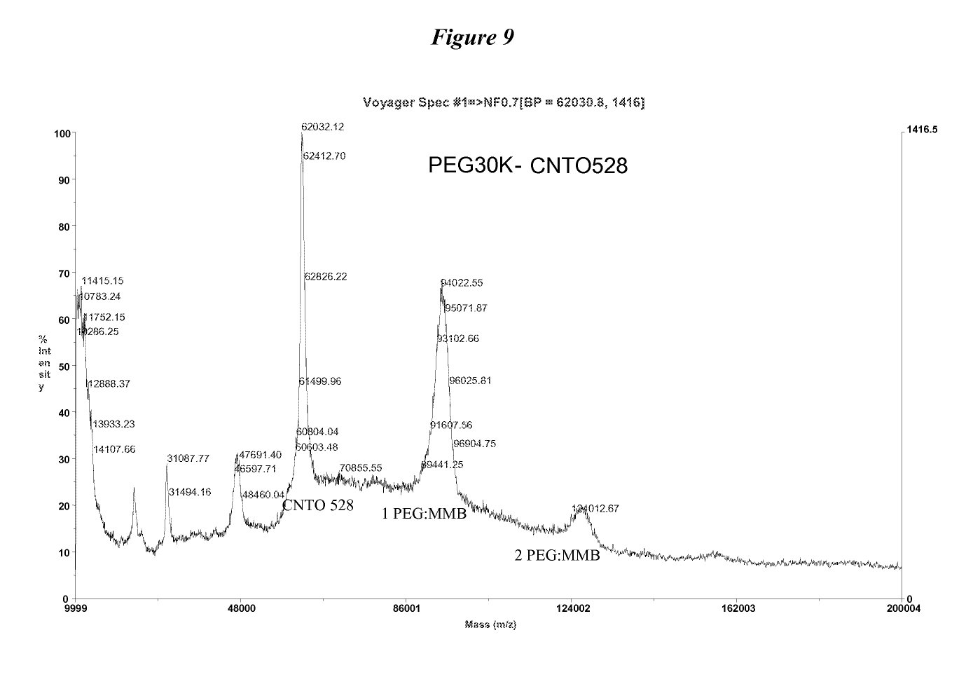

A MALDI-TOF-MS analysis of the conjugate was acquired using a Voyager DE instrument from Applied BioSystems (Foster City, Calif.). The sample was mixed with matrix solution (e.g. 1% sinnapinic acid in 50% acetonitrile:water containing 0.1% trifluoroacetic acid), loaded onto the target, and allowed to air dry. The trace is shown in FIG. 9. The peak at 62,032 m/z corresponds to unmodified CNTO528 and the peaks observed at 94,022 and 124,012 m/z correspond to a 1:1 and a 2:1 PEG-CNTO528 conjugate, respectively.

### Example 3

### UT-7 Cell Proliferation Assay

The biological activity of conjugates of PEG and CNTO528 was also assessed in vitro and in vivo.

A. Conjugate Preparation

PEG-CNTO528 conjugates were made and purified by a Protein A column and then tested by a UT-7 cell proliferation assay for bioactivity. Three conjugate preparations were prepared, as follows.

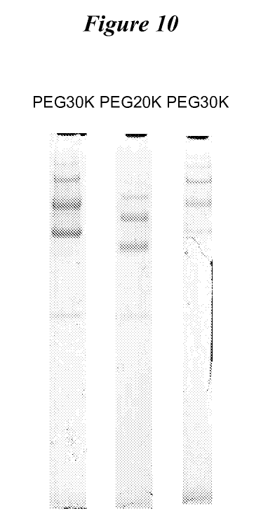

Conjugates of PEG-CNTO528 were prepared using PEG having a molecular weight of 30,000 Da or 20,000 Da. The ratio of PEG to CNTO528 was varied, to provide conjugates with varying extents of PEGylation. The extent of PEGylation was approximated using gel electrophoresis, as shown in FIG. 10.

1. Lightly PEGylated PEG₃₀ₖ-CNTO528 was prepared as in Example 1 and the reaction was stopped with 10 mM final glycine. Crude conjugation reactions were dialyzed to 50 mM sodium phosphate/150 mM NaCl pH 7.4 and loaded on a 1 mL HiTrap Protein A FF column (GE Healthcare). After washing the column in 100 mM Citrate, pH 5.0, the samples were eluted during a gradient to 100 mM Citrate, pH 3.5. Collected fractions were analyzed by SDS-PAGE for composition and then appropriate fractions were pooled and dialyzed to 50 mM sodium phosphate/150 mM NaCl pH 7.4. By SEC (Superose 6, 50 mM sodium phosphate and 150 mM NaCl, pH 7.4 mobile phase) the composition of this lightly PEGylated PEG₃₀ₖ-CNTO528 (lot# 040712A30KMUH) was 25.7% 3:1 and greater, 45% 2:1, 26.9% 1:1, and 2.3% free protein.

2. Heavily PEGylated PEG₃₀ₖ-CNTO528 was prepared similarly to lightly PEGylated PEG₃₀ₖ-CNTO528 except the conjugate reaction was at 25 molar excess of PEG₃₀ₖ-NPC and 30 mM final glycine was used to stop the reaction. The composition of heavily PEGylated PEG_{30K}-CNTO528 (lot# 040712B30KMUH) by SEC was 63.1% 3:1 and greater, 30.7% 2:1, 2.6% 1:1, and 3.6% free protein.

3. Lightly PEGylated PEG₂₀ₖ-CNTO528 was prepared using PEG₂₀ₖ-NPC (NM1101) at a 6-fold molar excess of PEG. The reaction proceeded as in Example 1 except the buffer composition was 25 mM MOPS/25 mM HOSu pH 7.0 final. The composition of lightly PEGylated PEG₂₀ₖ-CNTO528 (Lot# 040712A20KMUH) by SEC was 16.6% 3:1, 45.8% 2:1, 35.4% 1:1, and 2.2% free protein.

These three conjugate preparations were also characterized by gel electrophoresis (0.3-1.2 μg protein per lane) and stained with Coomassie™ blue stain for detection of protein. The gel is shown in FIG. 10, where the left lane corresponds to lightly PEGylated PEG₃₀ₖ-CNTO528, the center lane conjugate corresponds to lightly PEGylated PEG₂₀ₖ-CNTO528, and the right lane corresponds to heavily PEGylated PEG₃₀ₖ-CNTO528. The retardation in mobility due to increased molecule weights allows for the estimation of relative distribution of species in each preparation (PEG: protein of 3:1, 2:1, 1:1 or free CNTO528).

B. Proliferation Assay

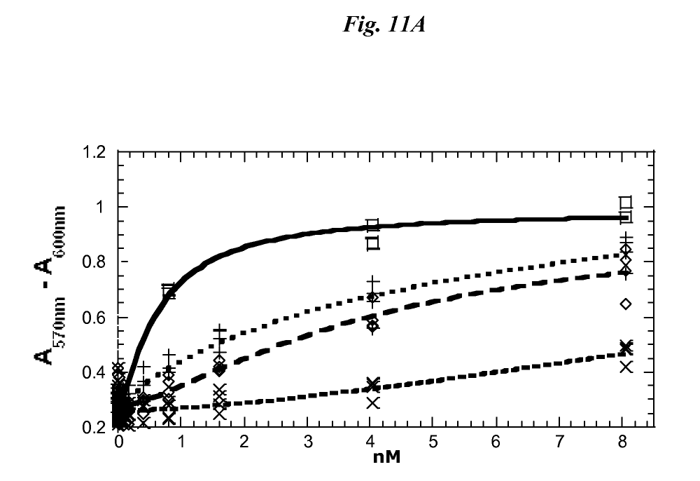

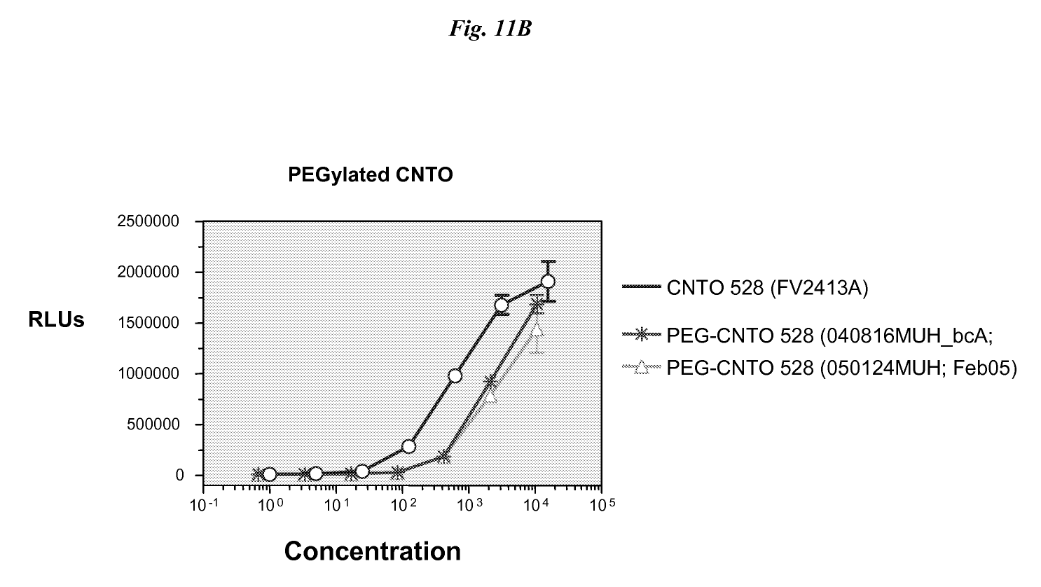

The bioactivity of PEG-CNTO528 conjugates was evaluated by a cell proliferation assay using an erythropoietin-dependent cell line, UT-7. Erythropoietin-dependent cells, UT-7 (Komatsu N., et al. (1993) Blood (1993) 82:456-64), were grown in the presence of erythropoietin (Epo) to 1×10⁶ cells/mL and then deprived of Epo for 24 hours. A sterile titration of one of the PEGylated CNTO528 conjugates was added to media-diluted cells in a 96-well plate. As a control, some cells were treated with unmodified CNTO528. Cells were grown 20 hours and then an appropriate amount of the metabolic dye AlmarBlue™ (Biosource, Inc.) was added. After 24 hours, the absorbance at 570 nm and at 600 nm was read (FIG. 11A) and the relative change for each concentration plotted (FIG. 11B) in order to fit to a dose-response curve.

Referring to FIG. 11A, UT-7 cells incubated with the Mimetibody™ CNTO528 alone (open squares) exhibited growth over the concentration range tested. The conjugates with a lower level of PEGylation, PEG_{30K}-CNTO528 conjugate (open diamonds) or PEG_{20K}-CNTO528 conjugate (+symbols), retained sufficient activity to permit growth of the cells, at a somewhat lower rate than for the naked Mimetibody™. The conjugate with a higher level of PEGylation (x symbols) showed a reduced bioactivity. Thus, the bioassay results of the various preparations showed that the CNTO528, which had an EC50 of 40 ng/mL in this assay (FIG. 11B) was reduced by PEGylation and, further, the relative loss in activity was a function of degree of PEGylation.

### Example 4

### Determination of Pharmacokinetic Parameters

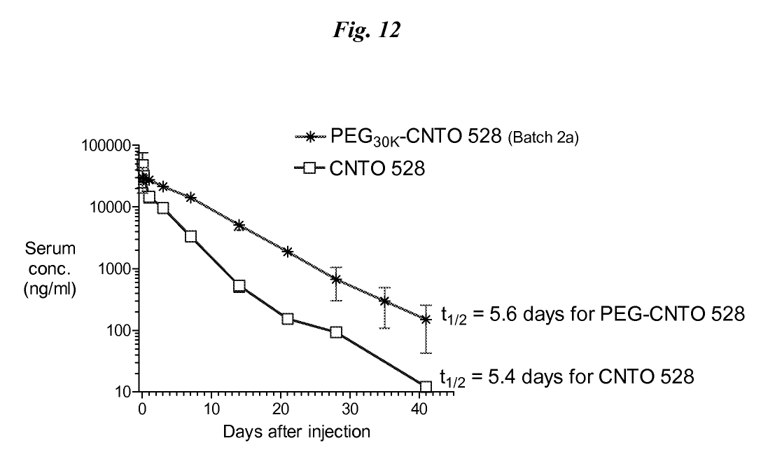

In vivo studies using rats and PEG_{30K}-CNTO528 were performed. In a first study to evaluate the pharmacokinetics of the conjugates, a PEG₃₀-CNTO528 was prepared according to Example 1. Rats were divided into two groups (n=4) for treatment intravenously with 1 mg/kg CNTO528 or PEG_{30K}-CNTO528. Sample bleeds were collected pre-dose, then at 1 hours, 5 hours, 24 hours, 72 hours, 7 days, 14 days, 21 days, 28 days, 35 days, and 41 days after injection. Serum samples were analyzed by an anti-huFc-specific ELISA, using the respective test article as standards for each group. It was expected that the more highly PEGylated species would remain in circulation longer than less PEGylated species and that these higher PEGylated species would not be as well detected by the ELISA assay (e.g., a 3:1 PEG:Mimetibody™ species would have a decreased interaction with the anti-huFc antibody relative to a 1:1 PEG:Mimetibody™ species). Thus, at later study timepoints, the amount of PEG₃₀ₖCNTO 528 may be underestimated. The results are shown in FIG. 12 and summarized in Table 7 below (end of specification).

The results are shown in FIG. 12. The pharmacokinetic parameters are reported in Table E below. Relative to unmodified CNTO528, PEG_{30K}-CNTO528 had greater than 2.5 times the exposure (AUC) and two times the mean residence time (MRT), as determined by Fc-specific measurements (Table 7).

### Example 5

### Determination of Hematopoietic Parameters

In this study, the pharmacodynamic response of a PEG₃₀ₖ-CNTO528, prepared as described in Example 2, was evaluated using rats and compared to the response achieved with unmodified CNTO528. Polyethylene glycol (PEG) of 30K average molecular weight was conjugated to CNTO528 by reductive amination, targeting accessible Lys residues (Lot# 050124MUH).

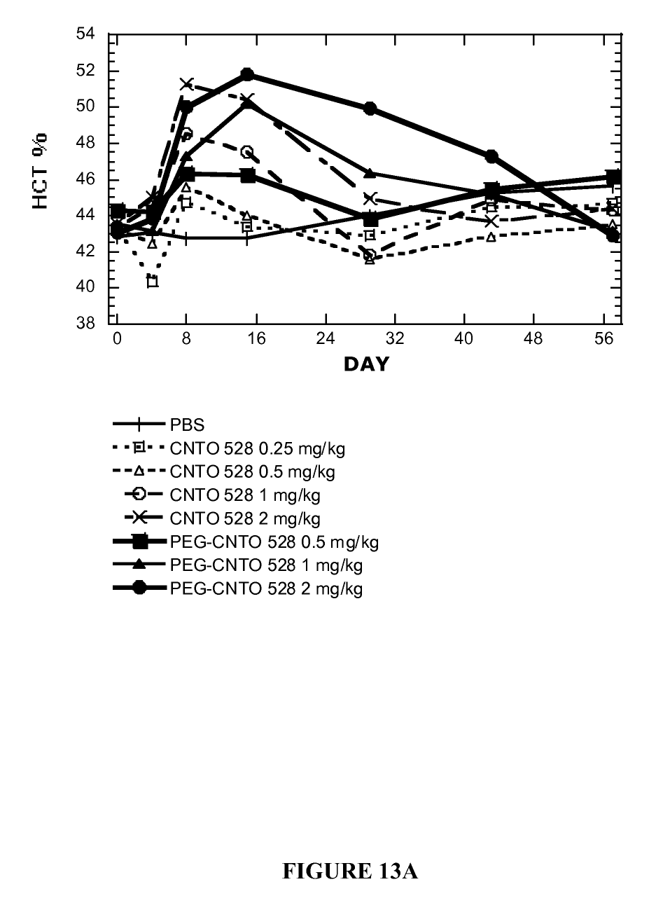

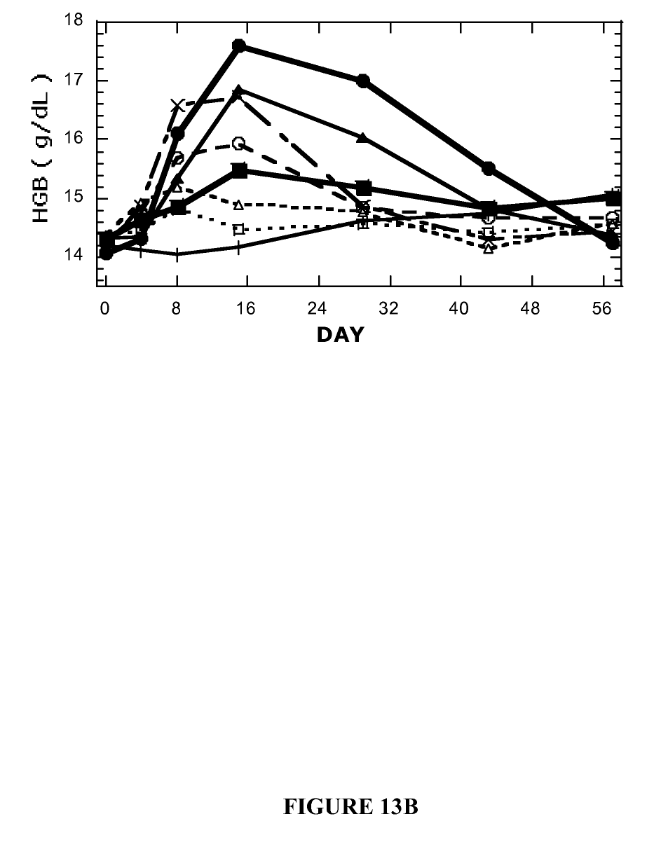

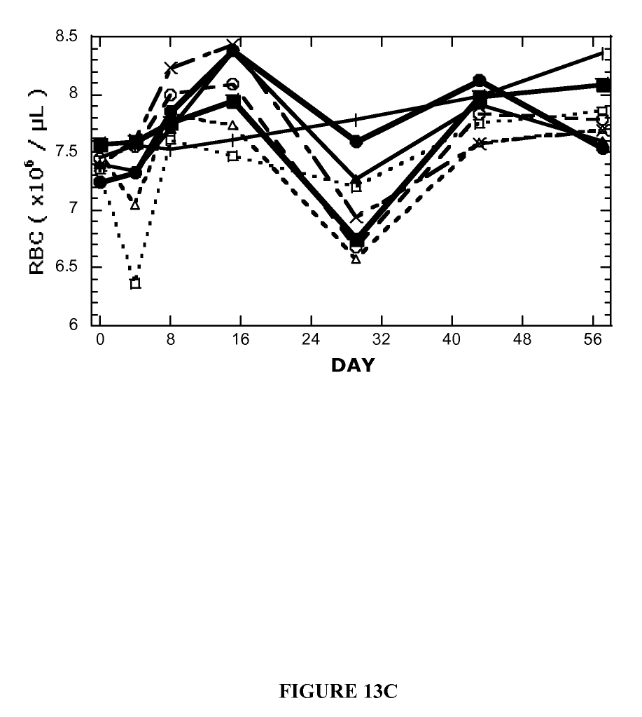

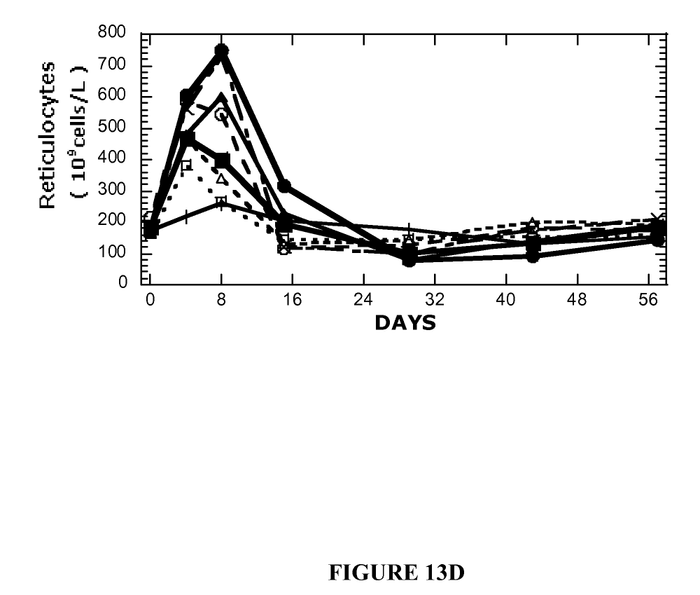

Forty (40) Female CD1 rats (approximately 300-350 grams) from Charles River Laboratories (Raleigh, N.C.) were randomized into eight test groups (n=5) for treatment with a single intravenous dose of PBS, CNTO528 (0.25, 0.5, 1, 2 mg/kg) or of PEG₃₀ₖ-CNTO528 (0.5 mg/kg, 1 mg/kg, 2 mg/kg). Whole blood hematology analysis was done pre-dosing and on days 4, 8, 15, 29, 43, and 57 including hematocrit, hemoglobin, RBC, reticulocytes. Blood was obtained by retro-orbital bleed on rats anesthetized using O₂CO₂ except on Day 57 when blood was obtained by cardiac puncture and prior to euthanasia via CO2 asphyxiation. Whole blood was used for analysis with the Advia 120 hematology analyzer (Bayer Diagnostics, Tarrytown, N.Y.). The results of the study are shown in FIGS. 13A-13D.

PEGylated CNTO528 caused a slightly slower onset of effects; but the level of stimulation was maintained at a higher level than for the corresponding dose of non-PEGylated CNTO528. By 16 days post-dosing, hemoglobin levels in rats treated with 2 mg/mL PEG30K-CNTO 528 were 17.6 g/dL, whereas the same dose of CNTO528 had only reached 16.7. More significantly, the duration of elevated Hb levels (FIG. 13B) from excursion until return to baseline was 50 days post-dosing for the PEGylated CNTO528 compared to 30 days for the unconjugated CNTO528. Thus, any loss of activity indicated from the in vitro cell assay did not translate to loss of pharmacodynamic response in animals, perhaps due in part to the superior pharmacokinetic profile of the PEGylated CNTO528.

### REFERENCES

J M Harris and S Zalipsky (1997) Poly(ethylene glycol) Chemistry and Biological Applications, ACS Symp. Series 680, Amer. Chem. Soc., Washington D.C.
Youngster S., et al (2002) “Structure, biology, and therapeutic implications of pegylated interferon alpha-2b.” Curr Pharm Des. 8(24):2139-57.
S M Chamow, et al. (1994) “Modification of CD4 immunoadhesin with monomethoxypoly (ethylene glycol) aldehyde via reductive alkylation.” Bioconjugate Chem. 5: 133-40.
Johnson, D. L., et al. (1998). “Identification of a 13 amino acid peptide mimetic of erythropoietin and description of amino acids critical for the mimetic activity of EMP1.” Biochemistry 37(11): 3699-710.
Komatsu N., et al.(1993). “Establishment and characterization of an erythropoietin-dependent subline, UT-7/Epo, derived from human leukemia cell line, UT-7.” Blood 82:456-64.

It will be clear that the invention can be practiced otherwise than as particularly described in the foregoing description and examples. Numerous modifications and variations of the present invention are possible in light of the above teachings and, therefore, are within the scope of the appended claims.

TABLE 1

| | IgG1 | IgG2 | IgG3 | IgG4 | IgM | IgA | IgE | IgD |
|---|---|---|---|---|---|---|---|---|
| Complement | ++ | + | +++ | − | +++ | + | − | − |
| Activation | | | | | | | | |
| Phagocyte | + | − | + | +/− | − | − | − | − |
| Binding | | | | | | | | |
| Neutral- | ++ | ++ | ++ | ++ | ++ | + | − | − |
| ization | | | | | | | | |
| Opsinization | +++ | +/− | ++ | + | − | + | − | − |
| Sensitization | ++ | − | ++ | − | − | − | − | − |
| for killing | | | | | | | | |
| by NKs | | | | | | | | |
| Sensitization | + | − | + | − | − | − | − | +++ |
| of Mast cells | | | | | | | | |
| Extra- | +++ | +++ | +++ | +++ | +/− | ++ | − | + |
| vascular | | | | | | (sIgA) | | |
| diffusion | | | | | | | | |

TABLE 2

| | SEQ ID | Upper Hinge | Hinge Core |
|---|---|---|---|
| Isotype | NO: | (length) | (length) |
| IgG1 | 1 | EPKSCDKTHT (10) | CPPCP (5) |
| IgG2 | 2 | ERK (3) | CCVECPPCP (9) |
| IgG3 | 3 | ELKTPLGDTTHT (12) | CPRCP(EPKSCDTPPPCPRCP)₃ (50) |
| IgG4 | 4 | ESKYGPP (7) | CPSCP (5) |

TABLE 3
Chromatogram Details for Crude Reaction Material (FIG. 6A)

| | Ret Time | % Peak | |
|---|---|---|---|
| Peak No | (min) | Area | PEG:MMB |
| 1 | 20.75 | 55.2 | 3:1 |
| 2 | 22.8 | 31.1 | 2:1 |
| 3 | 27.25 | 12.5 | 1:1 |
| 4 | 37.76 | 1.2 | free MMB |

TABLE 4
Chromatogram Details for Purified Reaction Material (FIG. 6B)

| | Ret Time | | % Peak | |
|---|---|---|---|---|
| Peak No | (min) | Peak Area | Area | PEG:MMB |
| 1 | 21.99 | 1497829 | 30.0 | 3:1 |
| 2 | 23.26 | 3455284 | 48.2 | 2:1 |
| 3 | 27.49 | 2448511 | 21.4 | 1:1 |
| 4 | 38.25 | 78898 | 0.5 | free MMB |

TABLE 5
Chromatogram Details for Crude Reaction Material (FIG. 7A)

| | Ret Time | % Peak | |
|---|---|---|---|
| Peak No | (min) | Area | PEG:MMB |
| 1 | 21.62 | 60.3 | 3:1 |
| 2 | 23.82 | 29 | 2:1 |
| 3 | 28.42 | 10.4 | 1:1 |
| 4 | 39.8 | 0.3 | free MMB |

TABLE 6
Chromatogram Details for Purified Reaction Material (FIG. 7B)

| | Ret Time | % Peak | |
|---|---|---|---|
| Peak No | (min) | Area | PEG:MMB |
| 1 | 22.51 | 20.0 | 3:1 |
| 2 | 24.03 | 46.2 | 2:1 |
| 3 | 28.23 | 32.7 | 1:1 |
| 4 | 39.58 | 1.1 | free MMB |

TABLE 7

| | | MRT | Cl | Vz |
|---|---|---|---|---|
| Sample | AUC × 10⁻³ | (day) | (mL/day) | (mL/kg) |
| PEG_{30K}-CNTO528 | 255 ± 4.1 | 72 ± 0.5 | 3.9 ± 0.1 | 30 ± 10 |
| CNTO528 | 95 ± 1.6 | 3.7 ± 0.3 | 10.7 ± 1.8 | 82.7 ± 16.8 |

What is claimed:

1. A method for increasing the circulating half-life of an Fc-containing protein by attaching a hydrophilic polymer, which method comprises:
a. contacting the Fc-containing protein with an activated carboxylic acid of a hydrophilic polymer in the presence of an activating agent at pH of less than about 7.0 or neutral pH, and
b. purifying said conjugated Fc-containing protein.

2. The method according to claim 1, wherein the hydrophilic polymer is selected from the group consisting of polyethylene glycol homopolymers, polypropylene glycol homopolymers, alkyl-polyethylene oxides, bispolyethylene oxides and co-polymers or block co-polymers of polyalkyene oxides.

3. The method according to claim 2, wherein the activated carboxylic acid of a hydrophilic polymer is mPEG-NPC.

4. The method according to claim 1, wherein the activating agent is HOSu, HOBt, and HOAt.

5. A conjugated Fc-containing protein made by the method of claim 1.

6. A conjugated Fc-containing protein made by the method of claim 3, wherein the ratio of PEG to polypeptide chain is 1:1 and the ratio of PEG to IgG heavy chain is 2:1.

7. A conjugated Fc-containing protein made by the method of claim 1 detectable in the blood of a subject, wherein the period in which said conjugate is detectable is the blood is sustained after administration of said conjugated Fc-containing protein for a greater period of time than that seen after administration of unconjugated Fc-containing protein.

8. A conjugated Fc-containing protein made by the method of claim 1 which retains a functional property of an Fc-domain selected from the group consisting of FcR binding, Protein A binding, protein G binding, Clq binding, ADCC, and CDC.

9. A conjugated Fc-containing protein of claim 5 or 6 having the biological properties of causing bone marrow cells to increase production of red blood cells.

10. A conjugated Fc-containing protein of claim 5 or 6 having the biological properties of causing 4 or 5 having the biological properties of stimulating the proliferation of UT7 cells.

11. A conjugated Fc-containing protein of claim 7 or 8 which is a PEG-conjugated CNTO528 (SEQ ID NO: 14) made by the method of claim 1.

12. A conjugated Fc-containing protein of claim 10 that causes bone marrow cells to increase production of red blood cells, and said increase is sustained after administration of said conjugated Fc-containing protein for a greater period of time than that seen after administration of unconjugated Fc-containing protein.

13. Any invention described herein.
